(19) United States
(12) Patent Application Publication
(10) Pub. No.: US 2021/0077190 A1
(43) Pub. Date: Mar. 18, 2021

(54) GENERATOR AND METHOD FOR AFFECTING BIOLOGICAL TISSUE AND CELLS USING MICROWAVE-INDUCED HEAT PROFILES

(71) Applicant: CENTRE NATIONAL DE LA RECHERCHE SCIENTIFIQUE, Paris (FR)

(72) Inventor: Maxim ZHADOBOV, Rennes (FR)

(73) Assignee: CENTRE NATIONAL DE LA RECHERCHE SCIENTIFIQUE, Paris (FR)

(21) Appl. No.: 17/048,288

(22) PCT Filed: Apr. 24, 2019
(86) PCT No.: PCT/EP2019/060504

§ 371 Date: Oct. 16, 2020

(30) Foreign Application Priority Data
Apr. 24, 2018 (EP) 18305507.8

(51) Int. Cl. A61B 18/18

(52) U.S. Cl. CPC A61B 18/1815; A61B 2018/00714; A61B 2018/1823

(57) ABSTRACT

A microwave generator configured to induce a change in temperature in a target area of a biological tissue so that the temperature of the target area exceeds the lethal threshold for the biological tissue, wherein the microwave generator is configured to release an electromagnetic pulse train in a frequency range between 0.4 GHz and 100 GHz that induces a thermal pulse train in the biological tissue, wherein: each pulse has a duration comprised between 100 ms and 2 minutes for the electromagnetic pulse train; the pulse width to period ratio is below 0.25 for the electromagnetic pulse train and the pulse width to period ratio is below 0.25 for the thermal pulse train; the peak to average ratio for the electromagnetic power exceeds 2 for the electromagnetic pulse train and the peak to average ratio for the temperature exceeds 2 for the thermal pulse train.

### FIELD OF INVENTION

The present invention relates generally to apparatus, systems, and methods for the thermal treatment of a biological tissue. In particular, the present invention relates to a system which is operable to induce increase in temperature in a biological tissue via an electromagnetic field emitted by a microwave generator.

### BACKGROUND OF INVENTION

In recent years, the quality of cancer detection and diagnostics has improved, but there remains a need for minimally invasive cancer treatments as an alternative to surgery, chemotherapy and radiotherapy to improve the efficiency of treatment and well-being of patients while reducing side effects and cost. Thermal therapies have been used to treat solid neoplasms inducing reversible or irreversible changes at cellular level. The aim of thermal treatment is to raise the temperature of pathological tissue without overexposing healthy tissue. It is essential to ensure necrosis of tumor cells within the desired volume of treatment and minimize thermal damage to healthy tissue surrounding the tumor. Heat sources used to increase the tumor temperature include radiofrequency, microwave, infrared, optical, ultrasound, and different kinds of hot sources (hot water, ferromagnetic seeds, nanoparticles, resistive implants).

Thermal therapy is understood to be the exposure of a patient to a higher temperature than their own body temperature. It is known in the art that higher temperatures can damage tumor cells while leaving normal tissue cells unharmed. Such application may either shrink or remove tumors from a patient and, in some instances, may be combined with other treatment options such as immunotherapy, chemotherapy and/or radiation to create a synergistic effect in treating the patient. A variety of different cancers may be treated with hyperthermic devices, a sample of which may include brain cancer, lung cancer, melanoma as well as additional other types.

Temperature-based treatments are subdivided into two groups with respect to the target tissue temperature. When the target temperatures are between 40° C. and 46° C., the term hyperthermia is used to describe the therapy (mild hyperthermia if the temperature delivered is between 40° C. and 43° C. and moderate hyperthermia between 43 and 46° C.). When tissue temperatures are above 50° C., the therapy is generally referred to as ablation. Except ablation of surface tumors with lasers, ablation is an invasive technique consisting in inserting the electrodes into the tissue to reach the tumor site. Usually it results in significant average heating of the tissue. The efficiency of thermal treatments of cancer for given biological model, physiological conditions and uniformity of the heat distribution over a target tumor region is determined by the cumulative thermal dose. The target ideal conditions of the currently used hyperthermia are typically defined as a spatially uniform constant dose over the tumor tissue volume, without overheating surrounding healthy tissue. The goals of the conventional hyperthermia operating with constant heating are mainly to boost the immune system and/or increase vasodilation at the tumor site.

It is known from the prior art international patent application WO 2010/151370, which discloses a method comprising the step of directing one or more pulses of electromagnetic radiation at the target. Said electromagnetic radiation pulses cause a temperature increase per unit of time in the biological tissue, and said temperature increase per unit of time causes a change of functioning in cells comprised in the biological tissue. The method disclosed in WO 2010/151370 produces a temperature increase per unit of time within a range of approximately one degree Celsius per second to approximately one degree Celsius per microsecond. However, the method disclosed in WO 2010/151370 does not deal with cumulative equivalent minutes (CEM), which is an indicator of the cell mortality. By decreasing the width to period ratio, CEM increases exponentially, and can exceed the lethal threshold while maintaining the average temperature at low level.

### SUMMARY

The present invention relates to a microwave generator configured to induce a change in temperature in a target area of a biological tissue so that the temperature of the target area exceeds the lethal threshold for the biological tissue, wherein the microwave generator is configured to release an electromagnetic pulse train in a frequency range between 0.4 GHz and 100 GHz that induces a thermal pulse train in the biological tissue, wherein:

each pulse has a duration comprised between 100 ms and 2 minutes for the electromagnetic pulse train;

the pulse width to period ratio is below 0.25 for the electromagnetic pulse train and the pulse width to period ratio is below 0.25 for the thermal pulse train;

the peak to average ratio for the electromagnetic power exceeds 2 for the electromagnetic pulse train and the peak to average ratio for the temperature exceeds 2 for the thermal pulse train.

According to one embodiment, the thermal pulse train in the target area of the biological tissue comprises a fraction inferior to 30% of thermal pulses having absolute peak temperature in a heat pulse exceeding 50° C. This feature advantageously prevents massive ablation of the biological tissue.

According to one embodiment, the microwave generator releases an electromagnetic pulse train in a frequency range between 20.1 GHz and 100 GHz. This sub-range is particularly advantageous due to fact that the penetration depth decreases and the power transmission coefficient at the skin/air interface increases for higher frequency values. Therefore, for a given incident power density, the energy transmitted in the biological tissue is absorbed in a smaller volume of the biological tissue so that the energy density is higher in said volume producing inside it a greater heating with a higher temperature gradient. Furthermore, using higher frequencies allows to easily generate shorter thermal pulses but with higher amplitude.

According to one alternative embodiment, the microwave generator releases an electromagnetic pulse train in a frequency range between 0.4 GHz and 9.9 GHz. This sub-range is advantageous since lower frequency penetrates deeper in biological tissues.

According to one embodiment, each pulse has a duration comprised between 600 ms and 2 minutes for the electromagnetic pulse train.

According to one embodiment, the pulse width to period ratio is comprised between 0.06 and 0.25 for the electromagnetic pulse train and the pulse width to period ratio is below 0.25 for the thermal pulse train. The advantage of these selections of values for the pulse width to period ratios (i.e. duty cycles) for the electromagnetic pulse train in combination with the selected parameters ranges is that of working in a CEM region exceeding the lethal threshold for the biological tissue while being in the range of practically achievable values.

According to one embodiment, the thermal pulse train is induced by an amplitude-modulated electromagnetic field.

According to one embodiment, the thermal pulses are induced by electromagnetic pulses.

According to one embodiment, the thermal pulse train comprises at least two alternating rise and drop intervals formed by electromagnetic power pulses.

According to one embodiment, the thermal pulse train is a sequence of thermal pulses induced by amplitude-modulated microwaves in one or several bands around at least one frequency in the following list of frequencies: {434 MHz, 915 MHz, 2.45 GHz, 5.8 GHz, 24 GHz, 61 GHz} corresponding to Industrial Scientific Medical (ISM) bands.

According to one embodiment, the microwave generator further comprises a radiating structure configured to emit an electromagnetic field inducing thermal pulses with a given heat distribution profile.

According to one embodiment, the microwave generator further comprises a clock control circuit configured to apply the thermal pulse train during a given duration.

According to one embodiment, the application of the thermal pulse train to the biological tissue comprised in one area targeted by the microwave generator generates a peak temperature in a heat pulse below 50° C.

According to one embodiment, the microwave generator further comprises a microwave power source comprising at least a power generator and/or power supply, a frequency synthesizer, a waveguide, an isolator, a regulator, a power divider and/or a power combiner.

According to one embodiment, the microwave generator further comprises a processor and a memory, wherein the memory comprises at least one table of correspondence comprising configuration data for selecting:

a duration of each electromagnetic pulse;

a thermal pulse width to period ratio; and/or

a thermal pulse peak to average ratio; said selection being compliant with a peak temperature in a heat pulse below 50° C. when the thermal pulse train is applied to the biological tissue comprised in one area targeted by the microwave generator.

The present invention further relates to a system configured to induce a change in temperature in a biological tissue, said system comprising a microwave generator according to any one of the embodiments described hereabove and a location module in order to generate position coordinates of a first area in the space, said coordinates being used to guide a waveform generator according to one orientation in order to produce a converging beam of the thermal pulse train in the first area.

According to one embodiment, the system further comprises a control unit of the microwave pulses comprising a control voltage and a current supply configured to modulate the amplitude of the electromagnetic field and of the generated thermal pulses.

According to one embodiment, the system further comprises a cooling system, which is applied in a nearby area of the first area during the generation of the thermal pulse train so as to contribute to the shaping of the thermal pulse and avoid overheating in the region surrounding the target area.

The present invention further relates to a method for inducing a change in temperature in a sample of a biological tissue, said method comprising:

identifying the location of at least one first area delimitating at least partially a target biological tissue;

guiding the orientation of a microwave generator according to any one of the embodiments described hereabove, so as to form a converging beam of the electromagnetic pulse in the first area; and

applying the electromagnetic pulse generating the thermal pulse train during a predefined duration.

According to one embodiment, the method further comprises the steps of:
selecting an emission mode comprising:
selecting a frequency mode;
selecting the waveform parameters;
selecting a width of each electromagnetic pulse;
selecting a pulse width to period ratio for the electromagnetic pulse train and the thermal pulse train;
selecting a peak to average ratio for the electromagnetic pulse train and the thermal pulse train;

controlling that said emission mode is compliant with a production of a temperature profile with a peak temperature in at least one heat pulse not exceeding 50° C. when the electromagnetic pulse train is applied in the first area.

The method according to the invention may be implemented using the microwave generator in all its configurations and the system in all its configurations, according to any one of the embodiments detailed in the present description.

The present invention further relates to a method for providing hyperthermia therapy to a target biological tissue comprising cancer cells, said method comprising:

identifying the location of at least one first area delimitating at least partially a target biological tissue with a location module configured to generate position coordinates of the first area;

using the coordinates of the first area, guiding the orientation of a microwave generator according to any one of the embodiments described hereabove, so as to form a converging beam of the electromagnetic pulse train in the first area;

applying the electromagnetic pulse train to the first area during a given duration so as to therapeutically treat the first area.

According to one embodiment, the method further comprises the steps of:

selecting an emission mode comprising:
selecting a frequency mode;
selecting waveform parameters;
selecting a width of each electromagnetic pulse;
selecting a pulse width to period ratio for the electromagnetic pulse train and the thermal pulse train;
selecting a peak to average ratio for the electromagnetic pulse train and the thermal pulse train;

controlling that said emission mode is compliant with a production of a temperature profile with a peak temperature in at least one heat pulse not exceeding 50° C. when the electromagnetic pulse train is applied in the first area

The present invention further relates to a method for providing hyperthermia therapy to a biological tissue comprising cancer cells, said method comprising the steps of:

providing a microwave generator configured so as to raise temperature of a target area of the biological tissue to achieve a therapeutic effect, wherein the microwave generator releases an electromagnetic pulse train in a frequency range between 0.4 GHz and 100 GHz that induces a thermal pulse train in the biological tissue, wherein:
each pulse has a duration comprised between 100 ms and 2 minutes for the electromagnetic pulse train (EPT);
the pulse width to period ratio is below 0.25 for the electromagnetic pulse train (EPT) and the pulse width to period ratio is below 0.25 for the thermal pulse train (TPT);
the peak to average ratio for the electromagnetic power exceeds 2 for the electromagnetic pulse train and the peak to average ratio for the temperature exceeds 2 for the thermal pulse train;

applying the electromagnetic pulse train (EPT) released by the microwave generator to a target area of the biological tissue so as to therapeutically treat the target area.

According to one embodiment, the microwave generator provided by the method releases an electromagnetic pulse train in a frequency range between 20.1 GHz and 100 GHz. This sub-range is particularly advantageous due to fact that the penetration depth decreases and the power transmission coefficient at the skin/air interface increases for higher frequency values. Therefore, for a given incident power density, the energy transmitted in the biological tissue is absorbed in a smaller volume of the biological tissue so that the energy density is higher in said volume producing inside it a greater heating with a higher temperature gradient. This feature is particularly advantageous when treating biological tissue on the surface of the patient comprising cancer cells such as melanoma.

According to one alternative embodiment, the microwave generator provided by the method releases an electromagnetic pulse train in a frequency range between 0.4 GHz and 9.9 GHz. This sub-range is advantageous since lower frequency penetrates deeper in biological tissues and therefore allows to reach biological tissue inside the patient and treat internal tumors.

According to one embodiment, the microwave generator provided by the method is configured to generate each pulse with a duration comprised between 600 ms and 2 minutes for the electromagnetic pulse train.

According to one embodiment, the method for providing hyperthermia therapy is provided to a biological tissue comprising cancer cells ex-vivo.

According to one embodiment, the pulse width to period ratio is selected between 0.06 and 0.25 for the electromagnetic pulse train and the pulse width to period ratio is below 0.25 for the thermal pulse train. The advantage of these selections of values for the pulse width to period ratios (i.e. duty cycles) for the electromagnetic pulse in combination with the selected parameters ranges is that of providing a hyperthermia therapy method working in a CEM region exceeding the lethal threshold for the biological tissue. On the contrary, it was shown that the use of duty cycle below 5% may be implemented in order to provide protective therapy for biological tissues or fluids having a chronic progressive disease, or a risk for a chronic progressive disease such as CPDs, including Type II Diabetes, Alzheimer's Disease, Idiopathic Pulmonary Fibrosis (IPF), heart disease and the like.

According to one embodiment, the microwave generator provided by the method is configured so that the thermal pulse train in the target area of the biological tissue comprises a fraction inferior to 30% of thermal pulses having absolute peak temperature in a heat pulse exceeding 50° C.

According to one embodiment, the microwave generator provided by the method is configured so that the thermal pulse train comprises at least two alternating rise and drop intervals formed by electromagnetic power pulses.

According to one embodiment, the microwave generator provided by the method is configured so that the thermal pulse train is a sequence of thermal pulses induced by amplitude-modulated microwaves in one or several bands around at least one frequency in the following list of frequencies: {434 MHz, 915 MHz, 2.45 GHz, 5.8 GHz, 24 GHz, 61 GHz} corresponding to Industrial Scientific Medical (ISM) bands.

According to one embodiment, the microwave generator provided by the method comprises a radiating structure configured to emit an electromagnetic field inducing thermal pulses with a given heat distribution profile.

According to one embodiment, the microwave generator provided by the method comprises a microwave power source comprising at least a power generator and/or power supply, a frequency synthesizer, a waveguide, an isolator, a regulator, a power divider and/or a power combiner.

According to one embodiment, the microwave generator provided by the method comprises a processor and a memory, wherein the memory comprises at least one table of correspondence comprising configuration data for selecting:

a duration of each electromagnetic pulse;

a thermal pulse width to period ratio; and/or

a thermal pulse peak to average ratio; said selection being compliant with a peak temperature in a heat pulse below 50° C. when the electromagnetic pulse train is applied to the biological tissue comprised in one area targeted by the microwave generator.

According to one embodiment, the microwave generator provided by the method comprises a location module in order to generate position coordinates of a first area in the space, said coordinates being used to guide a waveform generator according to one orientation in order to produce a converging beam of the electromagnetic pulse train in the first area.

According to one embodiment, the microwave generator provided by the method comprises a control unit of the microwave pulses comprising a control voltage and a current supply configured to modulate the amplitude of the electromagnetic field and of the generated thermal pulses.

According to one embodiment, the microwave generator provided by the method comprises a cooling system, which is applied in a nearby area of the first area during the generation of the thermal pulse train so as to contribute to the shaping of the thermal pulse and avoid overheating in the region surrounding the target area.

### DEFINITIONS

In the present invention, the following terms have the following meanings:

As used herein the singular forms “a”, “an”, and “the” include plural reference unless the context clearly dictates otherwise.

“Thermal treatment” and “Hyperthermia” refer to an increase in temperature above the normal human body temperature induced therapeutically.

“Heat profile” refers to the temperature dynamics as a function of time.

“Biological tissue”: refers to tissue as an ensemble of similar cells and their extracellular matrix from the same origin that together carry out a specific function. In present description “biological tissue” may as well refer to a group of cells or a solution comprising cells.

“Microwave” refers to an electromagnetic wave with frequency ranging from 300 MHz to 300 GHz.

“Biological tissue targeted” refers to a biological substance or structure that has to be affected, modified, or destroyed to achieve a desired biological effect. This includes, but is not limited to, biological cells (including cancer cells), sub-cellular structures and organelles, biological solution, biological tissue, malignant or benign tumors.

“Train of electromagnetic pulses” refers to a repetitive series of electromagnetic pulses, separated in time by a fixed and often constant interval. The duration of each pulse and its amplitude are also often constant.

### BRIEF DESCRIPTION OF THE DRAWINGS

FIG. 1 is a schematic representation of the invention according to one embodiment, wherein the microwave generator 1 releases an electromagnetic pulse train EPT that induces a thermal pulse train TPT in said biological tissue 2.

FIG. 2 shows the relationship between the cumulative equivalent minutes (CEM), computed for parameters given in the EXAMPLES, as a function of the ratio between the width of the thermal pulses and the period of the pulse train.

FIG. 3 is an illustrative representation of the waveform of the electromagnetic pulse train wherein only 3 pulses of in total 270 are represented. SAR refers to the Specific Absorption Rate.

FIG. 4 is a schematic representation of the experimental set-up presented in the EXAMPLE section of this description.

FIG. 5 is an illustrative representation of heat pulses measured at cellular level. Only 3 pulses of in total 270 are represented.

FIG. 6 is a histogram showing the cell survival rate for the continuously exposed melanoma cells and for the melanoma cells exposed to the electromagnetic train pulses. Said survival rate are obtained from the results of the experiments described in the EXAMPLE section of this description.

### REFERENCES

1 Microwave generator;

2 Biological tissue;

EPT Electromagnetic pulse train;

TPT Thermal pulse train.

### DETAILED DESCRIPTION

The following detailed description will be better understood when read in conjunction with the drawings. For the purpose of illustrating, the steps of the method and the device are shown in the preferred embodiments. It should be understood, however that the application is not limited to the precise arrangements, structures, features, embodiments, and aspects shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

Most of the chemical reaction rates behind cellular processes are transient and temperature sensitive (empirical relationship is provided by the Arrhenius law). Depending on parameters and conditions of heating, two mechanisms are at the origin of cellular responses including i) inactivation of protein functions and enzymatic activity, and ii) activation of signaling pathways. Protein and enzymatic inactivation are responsible for heat cytotoxicity and radio or chemo sensitization of the cells as responses to a severe heat shock (usually >43° C.), while induction of thermotolerance is the dominant activating response occurring when cells are exposed to sublethal temperatures, typically ranging from 39 to 42° C.

Thermotolerance is due to existence of protein quality control response, which is one of the most conserved cytoprotective mechanisms in evolution. In case of heat shock, cells overexpress chaperones and heat shock proteins (HSPs) that protect cellular proteins from misfolding and aggregation. HSPs have been identified as key determinants of cell survival because they also modulate apoptosis by directly interacting with components of the apoptotic machinery. These proteins are the key factors in response to cellular stress and they are involved in many pathologies such as cancer or neurodegenerative diseases. Their ability to bind to client proteins depends on their level of phosphorylation induced by heat shock response. HSP expression in cells may correlate with healing or may lead to tissue damage.

The pulsed electromagnetically-induced heating, as disclosed in the present invention, can lead to stronger damage in cells compared to continuous heating, allowing, in the case of thermo-oncological therapies, to decrease the treatment duration, reducing patient discomfort, and to eliminate or reduce the influence of blood perfusion as well as thermotolerance.

The present invention relates to a microwave generator 1 configured for inducing a change in temperature in a biological tissue 2. The present invention further relates to a treatment method for inducing a change in temperature. Currently used methods for conventional hyperthermia mainly produce a continuous and constant heat of the target biological tissue, i.e. tumor tissue. The method of the present invention, which is implementable using the microwave generator 1 of the present invention, uses an alternative approach consisting in a fractionation of the total duration of the electromagnetic radiation exposure in a plurality of time intervals. This approach results in the production of a train of electromagnetic pulses of an arbitrary shape. As for the continuous heating, the approach used in the present invention guarantee that the average temperature of the biological tissue 2, which has rose due to the electromagnetic pulses, remains inferior to the lethal threshold for biological tissue 2. However, the advantage of using a train of electromagnetic pulses is that the cumulative equivalent minutes (CEM) increases exponentially with decreasing the ratio between the width of the pulses and the period of the pulse train, potentially exceeding the lethal threshold for biological tissue 2, as showed by the curve in FIG. 2. This approach leads to an at least partial destruction of the cancer cells at low average heating, simultaneously reducing the damage to the surrounding healthy tissue. Furthermore, it prevents development of thermotolerance in cancer cells and tissue during thermal treatments further enhancing the treatment efficiency. In conventional hyperthermia (constant heating) the thermotolerance (i.e. enhanced synthesis of heat shock proteins leading to high resistance of cells to heat shock) results in an undesirable adaptive response of cancer cells reducing the efficiency of treatment. Note that this method can be also used to enhance the efficiency of tumor treatments when the average temperature and corresponding CEM exceed the lethal threshold.

According to one embodiment, the biological tissue 2 in which is induced the temperature change is a part of the human body or an animal body. According to an alternative embodiment, the biological tissue 2 is obtained from a biopsy or an in vitro cellular culture.

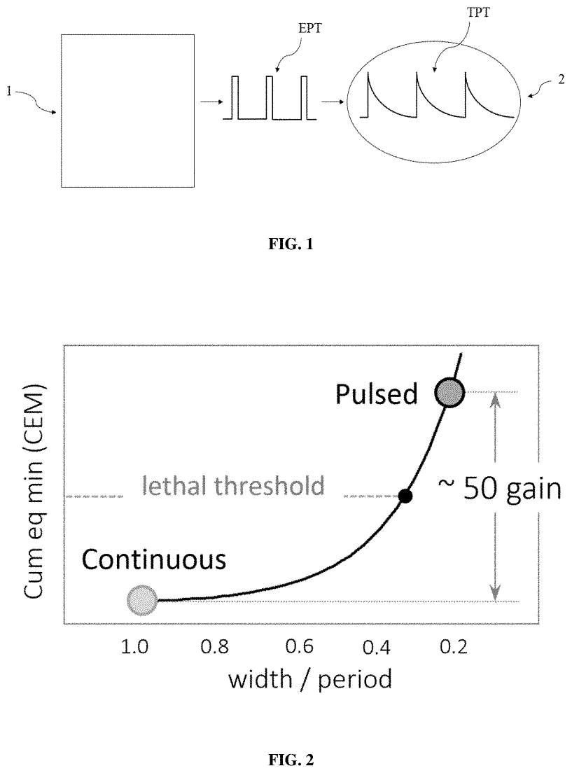

According to the embodiment illustrated in FIG. 1, the microwave generator 1 releases an electromagnetic pulse train EPT that induces a thermal pulse train TPT in the biological tissue 2 irradiated by said electromagnetic pulse train EPT. Said thermal train pulse TPT produces a heat profile in the biological tissue 2.

According to one embodiment, the microwave generator 1 comprises a power supply, at least one oscillator and at least one amplifier. In one embodiment, the microwave generator 1 comprises a magnetron and a modulator. The microwave generator 1 may comprise any component for modifying the waveform according to the desired transmitted output.

According to one embodiment, the heat profile is generated in a region defining the target biological tissue. Said target may be for example cancer cells or tissue, malignant or benign tumor or any other biological target that need to be treated or destroyed. According to one embodiment, the location and the two or three-dimensional delineation of the target region is determined from medical images obtained from one or more imaging techniques, such as MRI, CT scan, PET, SPECT, mammography, ultrasounds or any other suitable imaging technique known by the man skilled in the art.

According to one embodiment, the electromagnetic pulse train EPT is emitted in in the frequency range [0.4-100] GHz or in a frequency sub-range [0.4-9.9] GHz, in a frequency sub-range [20.1-50] GHz, in a frequency sub-range [20.1-100] GHz.

The embodiment consisting in releasing the electromagnetic pulse train EPT in a frequency sub-range between 20.1 GHz and 100 GHz, is particularly advantageous due to fact that the penetration depth decreases and the power transmission coefficient at the skin/air interface increases for higher frequency values. Therefore, for a given incident power density, the energy transmitted in the biological tissue is absorbed in a smaller volume of the biological tissue so that the energy density is higher in said volume and produces inside it a greater heating with a higher temperature gradient. Furthermore, using higher frequencies allows to easily generate shorter thermal pulses but with higher amplitude. This property of the upper part of the microwave spectrum is particularly advantageous when treating biological tissue on or close to the surface of the patient since, in addition to above mentioned advantages, it allows a higher resolution and greater precision in the delimitation of the target tissue during the treatment so as to spear the health tissue lying beneath or around the target tissue.

Inversely, the frequency sub-range between 0.4 GHz and 9.9 GHz is particularly advantageous due to the higher capability of penetration inside tissue at lower microwave frequencies. Therefore, the use of this sub-range is suitable for reaching biological tissue deep inside the patient so as to provide the hyperthermia therapy to treat internal tumors, according to the present invention.

In one embodiment, the electromagnetic pulse train EPT is emitted in the frequency band centered around 434 MHz, 915 MHz, 2.45 GHz, 5.8 GHz, 24 GHz or 61 GHz, corresponding to Industrial Scientific Medical (ISM) bands. The advantage of lower frequencies consists in the increased penetration depth of the electromagnetic field inside the biological tissue 2. However, the focusing resolution decreases. On the other hand, the advantage of higher frequency is that absorption in biological tissue 2 becomes more localized and focusing resolution increases. The power transmission to the biological tissue 2 at the air-to-biological tissue interface increases with frequency, as well. Note that above several GHz, surface overheating becomes an important issue, which can be partially eliminated by using enforced surface cooling. For example, the typical penetration depth into biological tissue is of the order of 5 cm, 1 cm, and 1 mm at 100 MHz, 1 GHz, and 50 GHz, respectively.

According to one embodiment, the electromagnetic pulse train EPT comprises at least two alternating rise and drop intervals forming electromagnetics pulses. According to one embodiment, the electromagnetic pulse train EPT comprises at least [2, 3, . . ., 10 000] alternating rise and drop intervals forming electromagnetics pulses.

According to one embodiment, the period of the electromagnetic pulse train (TPT) generating the heat pulses is constant. According to one embodiment, the period of the electromagnetic pulse train (TPT) generating the heat pulses is not constant.

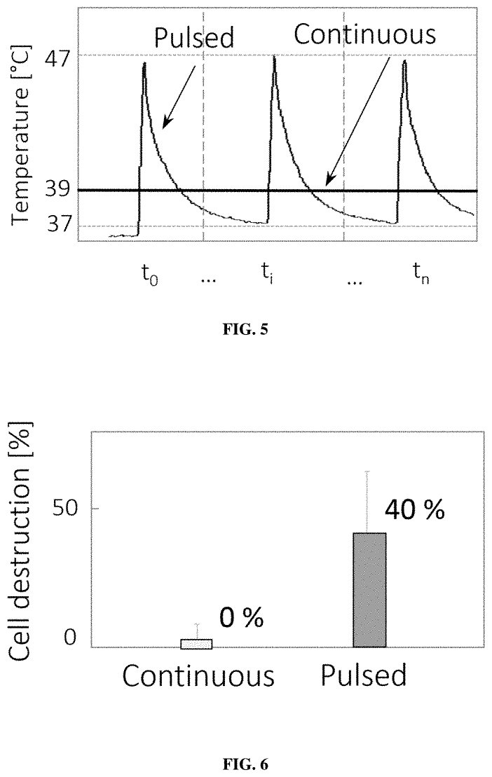

According to one embodiment, each pulse of the electromagnetic pulse train EPT has a duration comprised between 100 ms and 2 minutes, between 10 s and 1 minute, between 100 ms and 20 s or between 1 minute and 2 minutes. The advantage of having an electromagnetic pulse duration higher than 100 ms is that it induces a noticeable heating in a pulse needed to achieve the desired effect. However, in order to obtain such short pulse values, such as below 600 ms, high-power and costly microwave generators are required. On the other hand, the interest of using an electromagnetic pulse width not exceed approximatively 2 minutes is to avoid the development of thermotolerance in cells or biological tissue. Furthermore, longer durations, between 2 s and 2 minutes are more adequate to the use of lower frequencies. In a preferred embodiment, each pulse of the electromagnetic pulse train EPT has a duration comprised between 600 ms and 2 s, since they allow to generate thermal pulses having adequate amplitude (FIG. 5) for the given ranges of frequencies according to the application of the present invention. Here the electromagnetic pulse width is defined as the time interval between the moment (during the rise interval) when the amplitude of the pulse reaches 50% of the pulse peak power, and the moment the pulse amplitude drops (during the drop interval) to the same level (i.e. 50% of the pulse peak power).

According to one embodiment, the microwave generator 1 parameters are configured to be tuned in order to obtain a ratio between the thermal pulse width and the period of the thermal pulse train. According to this embodiment, the thermal pulse width of the electromagnetic pulse train and the period of the thermal pulse train are chosen to obtain a ratio inferior to a predefined threshold for electromagnetic pulse train EPT and thermal pulse train. Said period of the thermal pulse train is defined as the time interval between two consecutive heat pulses.

In one embodiment, said predefined threshold for electromagnetic pulse train EPT and thermal pulse train TPT ranges between 0.05 and 0.5, between 0.06 and 0.25, between 0.05 and 0.1, between 0.1 and 0.5, between 0.1 and 0.25 or between 0.25 and 0.5. In a preferred embodiment, said predefined threshold for electromagnetic pulse train EPT and thermal pulse train TPT is set at 0.25 or below. Given the ranges of frequencies and the duration of electromagnetic pulse train EPT, according to the preferred embodiment described hereabove, in order to generate thermal pulses having adequate amplitude for the application of the present invention, it is more advantageous to select the pulse width to period ratio superior to 0.06 for the electromagnetic pulse train EPT and the pulse width to period ratio superior to 0.06 for the thermal pulse train TPT. The range between 0.06 and 0.25 being therefore a preferred range for both above cited parameters.

The advantage of maintaining a ratio between the thermal pulse width and the period of the thermal pulse train below a predefined threshold for electromagnetic pulse train consists in obtaining an increment of the CEM significant enough to exceed the lethal threshold in the region defining the target biological tissue while maintaining the average temperature inferior to said lethal threshold.

According to one embodiment, the ratio between the pulse peak value and the average heat in at least one thermal pulse exceeds a predefined threshold. In one embodiment, said predefined threshold value ranges from 1 to 3. In one preferred embodiment said predefined threshold is set at 2 or above. In one illustrative example, the average temperature rise induced by at least one thermal pulse should not exceed half the value of the peak temperature of said thermal pulse.

The cumulative effect of the embodiment described hereabove produces the advantage of ensuring a gain in term of CEM compared to the constant continuous heating method with similar average temperature rise.

According to one embodiment, the absolute peak temperature of at least one thermal pulse exceeds 50° C. According to one embodiment, the fraction of thermal pulses, in one thermal pulse train, having an absolute peak temperature exceed 50° C. is inferior of a percentage comprised between 0 and 30%. The advantage of this embodiment is the prevention of massive ablation of the biological tissue targeted.

According to one embodiment, the microwave generator 1 is configured to be tuned to produce a peak power of the electromagnetic exposure so that the power density in the biological target induces a peak heating in at least one thermal pulse exceeding 3° C. According to one embodiment, the peak power is superior to 1 W.

According to one embodiment, the thermal pulse train is induced by a modulation of the amplitude of an electromagnetic field.

According to one embodiment, the thermal pulses are induced by non-sinusoidal periodic waveform. According to a preferred embodiment, the thermal pulses are induced by square waveform electromagnetic pulses. According to an alternative embodiment, the thermal pulses are induced by a sinusoidal, rectangular, triangle, sawtooth or similar waveform.

According to one embodiment, the microwave generator 1 further comprises a radiating structure configured to emit electromagnetic field inducing the thermal pulses with a predefined heat distribution profile. According to one embodiment, the radiating structure is an antenna or an antenna array such as a horn antenna, choker-ring antenna, planar structure, radial line slot antenna or the like. According to one embodiment, the microwave generator 1 further comprises connectors, adapters, and/or transitions needed to connect and match the antenna with the rest of the setup. Shaping the electromagnetic field in the target area using the above-mentioned antenna can be achieved by beam forming capabilities including lenses, reflectors, beam steering, matching layers, etc. According to one embodiment, the radiating structure is located at a predefined distance or is in direct contact with the target biological tissue 2.

According to one embodiment, the microwave generator 1 further comprises a clock control circuit which is an especially synchronous digital circuit, being configured to apply the thermal pulse train during a predefined duration. In one embodiment, circuits using the clock signal for synchronization become active at either the rising edge, falling edge, or, in the case of double data rate, both in the rising and in the falling edges of the clock cycle.

According to one embodiment, the microwave generator 1 comprises a microwave power source. In one embodiment, said microwave power source comprising at least a power generator and/or power supply, a frequency synthesizer, a waveguide, an isolator, a regulator, a power divider and/or a power combiner.

In the present invention the choices of each microwave generator parameters (i.e. frequency, pulse duration, pulse width to period ratio for the electromagnetic pulse train and the thermal pulse train, and peak to average ratio for the electromagnetic pulse train and the thermal pulse train) are highly interrelated to induce a change in temperature in a target area of biological tissue 2 so that the temperature of the target area exceeds the lethal threshold for the biological tissue. For example, it is possible to obtain equivalent heat distribution profiles at lower frequency when this change is counterbalanced by an increase of the incident power or an increase of the electromagnetic pulse duration. The choice of these values may further depend on the type of target biological tissue and its location.

According to one embodiment, the microwave generator 1 further comprises a processor and a computer readable memory. In one embodiment, the computer readable memory comprises at least one table of correspondence comprising configuration data for selecting:

a duration of each electromagnetic pulses.

a thermal pulse width to period ratio; and

a thermal pulse peak to average ratio.

According to one embodiment, the selection of these configurations is compliant with a peak temperature in a heat pulse below 50° C. when said thermal pulse train (TPT) is applied to biological tissue comprised in one area targeted by the microwave generator 1.

One aspect of the present invention relates to a system configured for inducing a change in temperature in a biological tissue. In one embodiment, said system comprises a microwave generator 1 according to the embodiment described hereabove. In one embodiment, the system further comprises a location module in order to generate position coordinates of at least a first area in the space, said coordinates being used to guide the waveform generator according to one orientation in order to produce a converging beam of the thermal pulse train (TPT) in the first area.

According to one embodiment, the system of the present invention further comprises a cooling system which is directly applied in a nearby area to the first area during the generation of the thermal pulse train. In one embodiment, when the biological tissue target is a near-surface tumor, enforced air flow, water circulation or another heat dissipation system can be applied to avoid undesired overheating of the region between the radiating structure and the target tissue.

The present invention further relates to a method for inducing a change in temperature in biological tissue.

According to one embodiment, the method for inducing a change in temperature in a sample of biological tissue ex-vivo.

In one embodiment, the method of the present invention comprises a preliminary step of identifying the location of at least a first area delimitating a target biological tissue. In one embodiment, said localization is performed on 2D or 3D medical images by automated computed implemented program for target delineation or by a member of the medical stuff. The images are obtained from medical imaging technics such as the ones described in an above embodiment.

In one embodiment, the method further comprises a step of guiding the orientation of the microwave generator 1 so to form a converging beam of the thermal pulse train TPT in the first area. In one embodiment, the orientation of the microwave generator 1 is generated by a treatment planning system. In one embodiment, the instruction for guiding for the orientation of the microwave generator 1 are outputted by said treatment planning system adapted for hyperthermia treatment.

In one embodiment, the method further comprises the step of applying the thermal pulse train during a predefined duration. In one embodiment, the duration of the thermal pulse train is comprised between 100 ms and 2 minutes.

According to one embodiment, the method further comprises a step of selecting an emitting mode. In one embodiment, said step of selecting an emitting mode comprises at least the selection of a frequency mode such as for example a frequency band for the electromagnetic pulse train EPT. In one embodiment, said step of selecting an emitting mode comprises at least the selection of waveform parameters such as the type of waveform (i.e. square, sinusoidal, etc.), the amplitude and the like. In one embodiment, said step of selecting an emitting mode comprises at least the selection of the width of each electromagnetic pulse. In one embodiment, said step of selecting an emitting mode comprises at least the selection thermal pulse width to period ratio, according to the embodiment described hereabove. In one embodiment, said step of selecting an emitting mode comprises at least the selection of a thermal pulse peak to average heat ratio, according to the embodiment described hereabove.

According to one embodiment, the method further comprises the step of controlling that said emitting mode is compliant with the prerequisite of producing of a temperature profile wherein the peak temperature in at least one heat pulse does not exceed 50° C. when said thermal pulse train will be applied in the first area.

Optional aspects of the present invention include the method of using the applicator for a variety of different ailments for the patient. One such optional use may be in the primary treatment of localized solid tumors. A similar but additional optional treatment may be in the adjuvant treatment of localized solid tumors in conjunction with either radiation or chemotherapy. Additionally, this treatment may also include for lymphoid tumors which can optionally include loco-regional disease.

While various embodiments have been described and illustrated, the detailed description is not to be construed as being limited hereto. Various modifications can be made to the embodiments by those skilled in the art without departing from the true spirit and scope of the disclosure as defined by the claims.

### EXAMPLES

The present invention is further illustrated by the following example.

Materials and Methods

Material

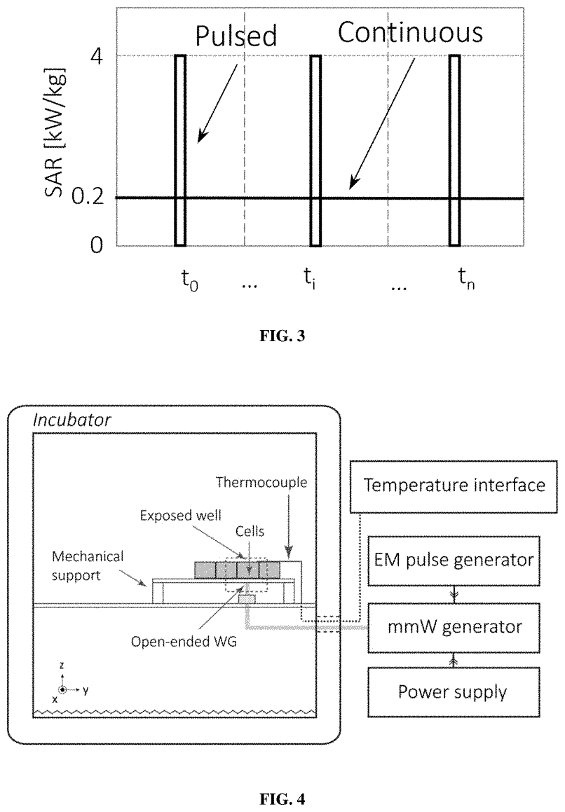

The experimental set-up, schematically represented in FIG. 4, consisted of:

a high-power millimeter-wave generator (Quinstar Technology, Torrance, Calif.) operating at 58 GHz having an output power up to 4.2W;

programmable power supply HMP 4040 (Hameg Instruments, Hampshire, UK) that provides control voltage and current for pulsed amplitude modulation of the millimeter-wave radiation;

open-ended rectangular WR-15 waveguide (aperture size is 3.81×1.905 mm²) used as an antenna;

a 12-well tissue culture plate (353072, Microtest 96, Becton Dickinson, Franklin Lakes, N.J.) with melanoma cells in culture (3 ml) used as a biological target.

a Thermocouple Reference design (Microchip Technology, Chandler, Ariz.) with sampling rate of 0.14 s;

K type thermocouple with the lead diameter of the probe of 75 mm (RS Components, Corby, UK).

Methods

The melanoma cells were exposed in vitro for 90 min to the pulsed amplitude modulated electromagnetic field at 58 GHz.

The melanoma cells exposed by the open-ended waveguide located 5 mm from the bottom of the tissue culture plate. The parameters of the pulsed amplitude modulated field and associated heating were the following: peak power 4 W, average power 0.2 W, electromagnetic pulse width of 1.5 s, period of 20 sec, width to period ratio of 0.075, peak temperature rise in a thermal pulse of ΔT_{p_max}=10° C., average temperature rise ΔT_{p_mean}≤2° C., and peak to average ratio in a thermal pulse of approximately 5. Normalized temporal waveform of electromagnetic pulses is shown in FIG. 3. Temperature was measured using the K type thermocouple with the lead diameter of the probe of 75 mm (RS Components, Corby, UK). To record temperature, was used the Thermocouple Reference design (Microchip Technology, Chandler, Ariz.).

In order to perform a comparison, a second culture plate of melanoma cells were continuously exposed with an electromagnetic field inducing a close average heating.

Multi-parametric microscopy analyses were performed to assess the survival rate. Other alternative techniques of the cell death and survival analysis can be used, employing for example cell death biomarkers. The experiments were independently reproduced three times.

Results

FIG. 2 illustrates CEM calculated as a function of the width-to-period ratio for heat pulses and continuous wave heating with an average temperature rise of 2° C. This estimation, obtained for exposure conditions provided in this example, clearly demonstrates the trend of the fast rise of CEM when the width-to-period ratio decreases. The lethal threshold level shown in FIG. 2 is indicative and depends on many parameters including cell type. The CEM curve demonstrates that, for the parameters considered here, cell mortality can be triggered for the width-to-period ratio <0.25.

The measured heating induced at cellular level by electromagnetic exposure is shown in FIG. 5.

As shown in FIG. 6, it was observed a reduction of the survival rate of melanoma cells after pulsed exposure compared to continuous heating resulting in the same average temperature rise. The survival rate of melanoma cells undergoing the continuous constant exposure was unchanged compared to non-exposed cells. The results of the three independent experiments were shown to be statistically significant using Anova test.

These exemplary results show the feasibility of the proposed invention. They demonstrate the feasibility of the destruction of cancer cells by thermal pulses induced by electromagnetic exposure with specific waveforms, without significant time-average heating of the biological target. Note that the observed effect is not limited to the frequency mentioned above (i.e. 58 GHz).

1-17. (canceled)

18. A microwave generator configured to induce a change in temperature in a target area of a biological tissue so that the temperature of the target area exceeds the lethal threshold for the biological tissue, wherein the microwave generator is configured to release an electromagnetic pulse train in a frequency range between 0.4 GHz and 100 GHz that induces a thermal pulse train in the biological tissue, wherein:
each pulse has a duration comprised between 100 ms and 2 minutes for the electromagnetic pulse train;
the pulse width to period ratio is below 0.25 for the electromagnetic pulse train and the pulse width to period ratio is below 0.25 for the thermal pulse train;
the peak to average ratio for the electromagnetic power exceeds 2 for the electromagnetic pulse train and the peak to average ratio for the temperature exceeds 2 for the thermal pulse train.

19. The microwave generator according to claim 18, wherein the thermal pulse train in the target area of the biological tissue comprises a fraction inferior to 30% of thermal pulses having absolute peak temperature in a heat pulse exceeding 50° C.

20. The microwave generator according to claim 18, wherein the thermal pulse train is induced by an amplitude-modulated electromagnetic field.

21. The microwave generator according to claim 18, wherein the thermal pulse train comprises at least two alternating rise and drop intervals formed by electromagnetic power pulses.

22. The microwave generator according to claim 18, wherein the thermal pulse train is a sequence of thermal pulses induced by amplitude-modulated microwaves in one or several bands around at least one frequency in the following list of frequencies: {434 MHz, 915 MHz, 2.45 GHz, 5.8 GHz, 24 GHz, 61 GHz} corresponding to Industrial Scientific Medical (ISM) bands.

23. The microwave generator according to claim 18, further comprising a radiating structure configured to emit an electromagnetic field inducing thermal pulses with a given heat distribution profile.

24. The microwave generator according to claim 18, further comprising a clock control circuit configured to apply the thermal pulse train during a given duration.

25. The microwave generator according to claim 18, further comprising a microwave power source comprising at least a power generator, power supply, a frequency synthesizer, a waveguide, an isolator, a regulator, a power divider and a power combiner.

26. The microwave generator according to claims 18, further comprising a microwave power source comprising at least a power generator, a frequency synthesizer, a waveguide, an isolator, a regulator and a power divider.

27. The microwave generator according to claim 18, further comprising a processor and a memory, wherein the memory comprises at least one table of correspondence comprising configuration data for selecting:
a duration of each electromagnetic pulse;
a thermal pulse width to period ratio; and/or
a thermal pulse peak to average ratio;
said selection being compliant with a peak temperature in a heat pulse below 50° C. when the electromagnetic pulse train is applied to the biological tissue comprised in one area targeted by the microwave generator.

28. A system configured to induce a change in temperature in a biological tissue, said system comprising a microwave generator according to claim 18 and a location module in order to generate position coordinates of a first area in the space, said coordinates being used to guide a waveform generator according to one orientation in order to produce a converging beam of the electromagnetic pulse train in the first area.

29. The system according to claim 28, further comprising a control unit of the microwave pulses comprising a control voltage and a current supply configured to modulate the amplitude of the electromagnetic field and of the generated thermal pulses.

30. The system according to claim 28, further comprising a cooling system, which is applied in a nearby area of the first area during the generation of the thermal pulse train so as to contribute to the shaping of the thermal pulse and avoid overheating in the region surrounding the target area.

32. A method for providing hyperthermia therapy to a biological tissue comprising cancer cells ex-vivo, said method comprising the steps of:
providing a microwave generator configured so as to raise temperature of a target area of the biological tissue to achieve a therapeutic effect, wherein the microwave generator releases an electromagnetic pulse train in a frequency range between 0.4 GHz and 100 GHz that induces a thermal pulse train in the biological tissue, wherein:
each pulse has a duration comprised between 100 ms and 2 minutes for the electromagnetic pulse train;
the pulse width to period ratio is below 0.25 for the electromagnetic pulse train and the pulse width to period ratio is below 0.25 for the thermal pulse train;
the peak to average ratio for the electromagnetic power exceeds 2 for the electromagnetic pulse train and the peak to average ratio for the temperature exceeds 2 for the thermal pulse train;
applying the electromagnetic pulse train released by the microwave generator to a target area of the biological tissue so as to therapeutically treat the target area.

33. The method according to claim 32, wherein the fraction of the pulses of the thermal pulse train in the target area of the biological tissue, which generates an absolute peak temperature in the pulse exceeding 50° C., is inferior to 30%.

34. The method according to claim 32, wherein the microwave generator is configured to be programmable so as to select an emission mode comprising:
selecting a frequency mode;
selecting waveform parameters;
selecting a width of each electromagnetic pulse;
selecting a pulse width to period ratio for the electromagnetic pulse train and the thermal pulse train;
selecting a peak to average ratio for the electromagnetic pulse train and the thermal pulse train.

35. A method for providing hyperthermia therapy to a target biological tissue comprising cancer cells, said method comprising:
identifying the location of at least one first area delimitating at least partially a target biological tissue with a location module configured to generate position coordinates of the first area;
using the coordinates of the first area, guiding the orientation of a microwave generator according to claim 18, so as to form a converging beam of the electromagnetic pulse train in the first area;
applying the electromagnetic pulse train to the first area during a given duration so as to therapeutically treat the first area.

36. The method according to claim 35, further comprising:
selecting an emission mode comprising:
selecting a frequency mode;
selecting waveform parameters;
selecting a width of each electromagnetic pulse;
selecting a pulse width to period ratio for the electromagnetic pulse train and the thermal pulse train;
selecting a peak to average ratio for the electromagnetic pulse train and the thermal pulse train;
controlling that said emission mode is compliant with a production of a temperature profile with a peak temperature in at least one heat pulse not exceeding 50° C. when the electromagnetic pulse train is applied in the first area.
